(19) United States
(12) Patent Application Publication
(10) Pub. No.: US 2011/0081329 A1
(43) Pub. Date: Apr. 7, 2011

(54) AMINO ACID COMPOSITIONS

(75) Inventors: Daniel S. Smith, Truckee, CA (US); Timothy Kuss, Centennial, CO (US); Julia Ross, Larkspur, CA (US)

(73) Assignee: Daniel S. Smith

(21) Appl. No.: 12/890,364

(22) Filed: Sep. 24, 2010

Related U.S. Application Data
(63) Continuation of application No. 11/811,224, filed on Jun. 7, 2007, now abandoned.
(60) Provisional application No. 60/804,155, filed on Jun. 7, 2006.

(51) Int. Cl. A61K 31/4172; A23L 1/30; A23L 1/302; A23L 1/304; A23L 1/305; A61K 38/43; A61P 3/04; A61P 25/00; A61P 25/24; A61P 25/22; A61P 29/02; A61P 25/30; A61P 25/32; A61P 25/20; A61P 3/08

(52) U.S. Cl. 424/94.1; 426/72; 426/2; 514/400

(57) ABSTRACT

Compositions are described herein that are nutritional supplements that contain free amino acids. These supplements contain a homogenous mixture of free amino acids, wherein the free-form amino acids comprise L-Lysine, L-Valine, L-Tryptophan, L-Phenylalanie, L-methionine, L-Leucine, L-Threonine, L-Isoleucine, L-Arginine, L-Histindine, L-Tyrosine, L-Carnitine, L-Serine, L-Glutamine, Aspartic Acid, L-Proline, L-Glycine, Taurine, L-Cysteine, Gamma-aminobutyric acid (GABA), L-Alanine, L-Glutamic acid, and wherein the composition comprises at least one B vitamin. These compositions are of use to treat disorders associated with a deficiency at least one amino acid, such as soft tissue injury, insomnia, panic attacks, anxiety disorders, eating disorders, anorexia, depression, chronic pain, emotional distress, chemical dependence, alcoholism, and hypoglycemia.

### PRIORITY CLAIM

This is a continuation of U.S. patent application Ser. No. 11/811,224, filed on Jun. 7, 2007, which in turn claims the benefit of U.S. provisional application 60/804,155 filed on Jun. 7, 2006. Both of the prior applications are incorporated herein by reference in their entirety.

### FIELD

This specification relates to the field of dietary supplements, especially to optimal blends of free amino acids and their use.

### BACKGROUND

Nutritional compositions have been the subject of intense research in recent years as investigators have begun to appreciate the beneficial health effects they can provide. Although many nutritional supplements have been devised to assist in exercise and bodybuilding regimes, such supplements have also been widely used to help improve cognition, memory and mood, and counteract the effects of cardiovascular disease, endocrine disorders, metabolic abnormalities, any many other conditions.

Amino acids are the structural units of proteins, which play crucial roles in virtually all biological processes. Some of the scientific research in the nutritional field has focused on the use of free amino acids to provide health benefits. The Amino Revolution (Simon & Schuster, 1987) by Robert Erdmann proposed that various combinations of free amino acids could help stimulate immunity, diminish heart disease, reduce anxiety, relieve depression and facilitate weight loss. The Mood Cure (Penguin Books, 2002) by Julia Ross described nutritional therapy regimes that were useful to improve emotional health by combating depression, anxiety, irritability, stress, eating disorders and other conditions. Amino Acids in Therapy (Healing Arts Press, 1985) by Leon Chaitow had proposed using an HPLC profile of a person's amino acid levels in blood or urine to detect deficiencies of certain amino acids or abnormalities in metabolic pathways that could affect health. It has been proposed that certain amino acids can be selectively supplemented to offset deficiencies and provide specific health benefits.

Although the beneficial effects of amino acids have been reported in the scientific literature, the ideal sources and combinations of nutrients have been the subject of substantial dispute. In the past, meat and eggs had been touted as excellent sources of protein to maintain health. However the cholesterols and other fats in these animal-based dietary sources have in recent years been found to adversely affect lipid profiles of many people who consume them. Conventional medical practice has therefore advised limiting dietary intake of these sources of protein.

An additional nutritional problem is presented by modern agricultural practices, which provide protein sources to animals that are deficient in certain important nutrients. If feed sources contain undesirably low levels of important amino acids, lower than desired levels of those amino acids are present in protein sources obtained from the animal. The unusual circumstances of modern life have also provided stresses and physiological challenges that may not be met by prior food sources.

### SUMMARY

Compositions are described herein that are nutritional supplements that contain free amino acids. These supplements contain a homogenous mixture of free amino acids, wherein the free-form amino acids comprise L-Lysine, L-Valine, L-Tryptophan (or 5-Hydroxytryptophan), L-Phenylalanie, L-methionine, L-Leucine, L-Threonine, L-Isoleucine, L-Arginine, L-Histindine, L-Tyrosine, L-Carnitine, L-Serine, L-Glutamine, Aspartic Acid, L-Proline, L-Glycine, Taurine, L-Cysteine, Gamma-aminobutyric acid (GABA), L-Alanine, L-Glutamic acid, and wherein the composition comprises at least one B vitamin.

These compositions are of use to supplement the nutrition of a subject, including healthy subjects and subjects with a condition associated with a relative deficiency of one or more amino acids. Exemplary conditions include soft tissue injury, insomnia, panic attacks, anxiety disorders, eating disorders, anorexia, depression, chronic pain, emotional distress, chemical dependence, alcoholism, and hypoglycemia. The composition can be administered to a subject one or more times.

The foregoing and other objects, features, and advantages of the invention will become more apparent from the following detailed description, which proceeds with reference to the accompanying figures.

### DETAILED DESCRIPTION

### Terms

“Administering” a dosage or dosage form includes self-administration by the subject, administration by another to the subject, and providing advice for administration to the subject (as in instructions provided in a tangible medium, such as printed instructions or advice on a computer readable medium). Administration by another to the subject can include, for example, administration by a physician, nurse or other health care provider or dietary consultant (such as a nutritionist). Administration also includes providing an end product (such as a solid composition in tablet or powdered form or a liquid composition) that is consumed or utilized by the subject.

“Ameliorating” or “ameliorate” refers to any indicia of success in the treatment of a pathology or condition, including any objective or subjective parameter such as abatement, remission or diminishing of symptoms or an improvement in a patient's physical or mental well-being. Amelioration of symptoms can be based on objective or subjective parameters; including the results of a physical examination and/or a psychiatric evaluation. For example, amelioration of symptoms can be measured by diagnostic tests, such as blood tests. In addition, amelioration of symptoms can be measured by psychiatric evaluation. For example a clinical guide to monitor the effective amelioration of a psychiatric disorder, such as depression, is found in the Structured Clinical Interview for DSM-IV Axis I mood disorders (“SCID-P”) (see fourth edition of Diagnostic and Statistical Manual of Mental Disorders (1994) Task Force on DSM-IV, American Psychiatric Association (“DSM-IV”); Kaplan, Ed. (1995); Comprehensive Textbook of Psychiatry/VI, vol. 1, sixth ed., pp 621-627, Williams & Wilkins, Baltimore, Md.).

“Amino acids” are the structural units of proteins. The twenty amino acids encoded by the genetic code are called “standard amino acids.” These amino acids have the structure H₂N—CHR—COOH, where R is a side chain specific to the amino acids. Standard amino acids are Alanine, Arginine, Aspargine, Aspartic acid, Cysteine, Glutamic acid, Glutamine, Glycine, Histidine, Isoleucine, Leucine, Lysine, Methionine, Phenylalanine, Proline, Serine, Theroinine, Tryptophan, Tyrosine, and Valine. In addition to the twenty standard amino acids, there are two additional amino acids called selenocysteine and pyrrolyne. “Non-standard amino acids” are additional amino acids that are not incorporated into proteins. These include the sulfur-containing Taurine and the neurotransmitter Gamma-aminobutyric acid (GABA). Other examples are Lanthioinine, 2-Aminoisobutyric acid, Dehydroalanine, Carnitine, Ornithine and Citrulline. An “essential” amino acid is an amino acid that cannot be synthesized by an organism, such as a human, and must therefore be obtained in the diet. For humans, the essential amino acids are Isoleucine (Ileu), Leucine (Leu), Lysine (Lys), Methionine (Met), Phenalanine (Phe), Threonine (Thr), Tyrosine (Try), and Valine (Val).

A “free” or a “free-form” amino acid is not covalently bonded to any other amino acid, and thus is not included in a protein or polypeptide. A free amino acid can generally be absorbed into the bloodstream without digestion. Chemical derivatives of a free amino acid that can substitute for the amino acid in a biochemical process are also encompassed by this disclosure. Thus, the amino acids can be used in the form of metal salts such as sodium salt, potassium salt, inorganic acid salts such as hydrochloride, sulfate, or organic acid salts such as acetate, lactate, malate. There are cases in which the above-mentioned amino acids partly or wholly may be used in the form of N-acetyl derivatives, such as N-acetyl derivative and the like. The amounts of amino acids expressed herein are in the form of the free base, unless indicated otherwise in a specific example.

Amino acids can be present in two stereoisometric forms, called “D” and “L.” The D and L form of any amino acid have identical physical properties and chemical reactivities, but rotate the plane of plane-polarized light equally but in opposite directions and react at different rates with asymmetric reagents. Most enzymes acting upon amino acids have asymmetric binding sites and thus can discriminate between the D and L forms. All naturally occurring amino acids in proteins are in the L form, although D-amino acids are found in some living cells, such as in the cell walls of microorganisms.

An “anxiety disorder” is a disorder characterized by a pattern of frequent, persistent worry about several different events or activities. The symptoms generally last at least six months. Generally the anxiety is out of proportion to the impact of the event or circumstance that is the focus of the worry.

“Chemical dependence” is a strong dependency on a substance that it becomes necessary to have this substance to function properly. Generally, alcoholism and drug addiction (such as to methadone, heroine, methamphetamines, or cocaine) are two forms of chemical dependence.

“Chronic pain” is mild to severe pain that lasts over an extended period of time. Generally, it is pain that persistent for longer than the average period of pain associated with a particular injury or condition.

“Depression” is a mental state of depressed mood characterized by feelings of sadness, despair and discouragement. Depression includes feelings of sadness considered to be normal (mild depression), dysthymia, and major depression. Depression can resemble the grief and mourning that follows bereavement, and there are often feelings of low self esteem, guilt and self reproach, withdrawal from interpersonal contact and somatic symptoms such as alterations in eating habits and sleep disturbances. A “mood disorder” is any of several psychological disorders characterized by abnormalities of emotional state and including especially major depressive disorder, dysthymia, and bipolar disorder.

A “dosage form” comprises any preparation, or combination of preparations, that provides a desired dosage. Hence a dosage form can include a single composition (such as a capsule or other ingestible preparation) or a combination of several different compositions (such as a powdered beverage mix, a tablet and/or a liquid supplement). A dosage form can “provide a daily dosage” in either a single unit dosage form (such as a tablet or a powder) or in multiple dosages taken at different times throughout a day. Hence a dosage form that includes multiple sub-dosage forms can provide the total daily dosage administered at different times during a day (for example twice or three times a day), and in different forms (for example as a liquid beverage and a tablet).

An “eating disorder” is an emotional disorder that manifests itself in an irrational craving for, or avoidance of, food. Two examples of eating disorders are bulimia and anorexia nervosa. “Anorexia” is characterized by an uncontrolled lack of appetite for food.

“Emotional distress” is a highly unpleasant emotional reaction (as anguish, humiliation, or fury) generally resulting from actually occurring events.

“Fibromyalgia” is a disorder characterized by muscle pain, stiffness and easy fatigability. The American College of Rheumatology has established diagnostic criteria that include pain on both sides of the body, both above and below the waist, as well as in an axial distribution (cervical, thoracic, or lumbar spine or anterior chest). In addition there must be point tenderness in at least 11 of 18 specified sites.

A “homogenous mixture” is a formulation of ingredients mixed together as a single composition, such that the active ingredients are all admixed with a carrier.

“Hypoglycemia” is also known as low blood sugar (glucose). When symptoms of hypoglycemia occur together with blood glucose level under 45 mg/dl for a human subject, and the symptoms promptly resolve with the administration of glucose, the diagnosis of hypoglycemia can be made with some certainty. Symptoms of hypoglycemia can include anxiety, sweating, tremor, palpitations, nausea, and pallor. Lack of glucose to the brain can cause symptoms ranging from headache, mild confusion, and abnormal behavior, to loss of consciousness, seizure, and coma. The causes of hypoglycemia include drugs (such as insulin), liver disease, surgical absence of the stomach, tumors that release excess amounts of insulin, and pre-diabetes. In some patients, symptoms of hypoglycemia occur during fasting (fasting hypoglycemia). In others, symptoms of hypoglycemia occur after meals (reactive hypoglycemia).

“Insomnia” is an abnormal wakefulness characterized by an inability to sleep, for example over a period of time of more than 24 hours. Insomnia can be classified as transient (short term), intermittent (on and off), and chronic (constant). Insomnia lasting from a single night to a few weeks is referred to as transient. If episodes of transient insomnia occur from time to time, the insomnia is said to be intermittent. Insomnia is considered to be chronic if it occurs on most nights and lasts a month or more.

A “panic disorder” is a condition wherein an individual has recurrent panic attacks. The panic attacks usually begin abruptly and include rapid heartbeat, chest sensations, shortness of breath, dizziness, tingling, and anxiousness.

“Percent by weight” is the weight of a single ingredient divided by the weight of the total amount of ingredients, expressed as a percentage. “Percent by weight of active ingredients” is the weight of a single active ingredient divided by the total weight of active ingredients. “Active” ingredients include amino acids, vitamins, minerals, co-factors and other therapeutically effective ingredients. “Inactive” ingredients are those ingredients that do not have a therapeutic effect, such as solvents, carriers, preservatives and flavoring agents.

A “powder” or an “ingestible powder” refers to a dry mixture that is suitable for mixing in a liquid base (such as water or milk) to provide a beverage in which the powder is dispensed to a subject.

“Pharmaceutically acceptable carriers” are any conventional pharmaceutically acceptable carriers useful in this invention are conventional. Remington's Pharmaceutical Sciences, by E. W. Martin, Mack Publishing Co., Easton, Pa., 15th Edition (1975), describes compositions and formulations suitable for pharmaceutical delivery of the fusion proteins herein disclosed.

In general, the nature of the carrier will depend on the particular mode of administration being employed. In addition to biologically-neutral carriers, pharmaceutical compositions to be administered can contain minor amounts of non-toxic auxiliary substances, such as wetting or emulsifying agents, preservatives, and pH buffering agents and the like, for example, sodium acetate or sorbitan monolaurate.

“Physiologically effective amount” refers to an amount of an agent that is capable of providing a desired physiological effect. For example, in some embodiments, of the disclosed compositions there is a sufficient amount of amino acids and vitamins to have a beneficial effect on the subject, for example by improving the subject's mood, cognition, anxiety level, ability to sleep, appetite, fibromyalgia, decreasing pain, or another condition that this ameliorated by the composition. An effective amount can ameliorate a sign or a symptom of a condition.

A “soft tissue injury” is damage of one or more tissues that connect, support, or surround other structures and organs of the body. Soft tissue includes muscles, tendons, fibrous tissues, fat, blood vessels, nerves, and synovial tissues; the four basic classes of tissues affected are the epithelial, muscular, nervous and connective tissues. These types of injuries are a major source of pain and disability. Soft tissue injuries include sprains, strains, subluxation, repetitive stress injury, and carpal tunnel syndrome.

“Subject” refers to any mammal. In one embodiment, a subject is a human subject.

A “supplement” refers to a non-food form of dosage administration. An example of a supplement is a pharmaceutical preparation (such as a tablet, enteral liquid, parenteral liquid, capsule, intranasal liquid or other form). In a particular disclosed example the supplement is a pharmaceutical preparation, in particular a tablet or capsule. However, a supplement can be administered in any form, including, but not limited to, ingredients formulated for transdermal, intravenous, subcutaneous, intra-muscular, or sublingual delivery.

“Vitamins” are organic molecules that are needed in small amounts in the diet. Humans do not have the capacity to synthesize vitamins, and thus they are required in the diet. Vitamin B2 is also known as riboflavin. Vitamin B2 is a water-soluble vitamin that is involved in energy production by aiding in fat, carbohydrate, and protein metabolism. Vitamin B2 is required for red blood cell formation and respiration, antibody production, and for regulating human growth and reproduction. Vitamin B3 is also known as Niacin or nicotinic acid, and includes the amide form, nicotinamide or niacinamide. Vitamin B3, is a water-soluble vitamin whose derivatives such as NADH NAD, NAD⁺, and NADP play essential roles in energy metabolism in the living cell and DNA repair. Severe lack of niacin causes the deficiency disease pellagra, whereas a mild deficiency slows down the metabolism, which in turn decreases cold tolerance and is a potential contributing factor towards obesity. Vitamin B6 is also known as pyridoxine, pyridoxal, and pyridoxamine, which are converted to pyridoxal 5′-phosphate (PLP). Vitamin B6 is a cofactor in many reactions of amino acid metabolism. PLP also is necessary for the enzymatic reaction governing the release of glucose from glycogen. Inadequate levels of vitamin B6 can impair nerve function and mental health. Vitamin B12 (cyanocobalamin, molecular formula C₆₃H₈₈CoN₁₄O₁₄P), is important for converting fats, carbohydrates, and protein into energy and assisting in the synthesis of red blood cells. B₁₂ deficiency is the cause of several forms of anemia.

### Description of Several Embodiments

Compositions are disclosed herein that meet the needs of many subjects, and thus serve as nutritional supplements. The subjects can be healthy. However, the subject can also have a condition associated with a relative deficiency of one or more amino acids, including conditions such as, but not limited to, soft tissue injury, eating disorders, anorexia, depression, chronic pain, emotional distress, chemical dependence, alcoholism, and hypoglycemia. These compositions provide essential free and non-essential free amino acids, and include all twenty standard free amino acids. The compositions include all of the standard free amino acids (including all of the essential free amino acids), as well as at least one B vitamin. The supplements are formulated for any route of administration, including but not limited to enteral administration, such as oral administration.

Thus, in one embodiment, the composition includes a homogenous mixture of free amino acids, or one or more therapeutically effective derivative(s) of an amino acid, or one or more metabolites of an amino acid. The homogenous mixture includes L-Lysine, L-Valine, L-Tryptophan, L-Phenylalanie, L-Methionine, L-Leucine, L-Threonine, L-Isoleucine, L-Arginine, L-Histindine, L-Tyrosine, L-Carnitine, L-Serine, L-Glutamine, Aspartic Acid, L-Proline, L-Glycine, Taurine, L-Cysteine, Gamma-aminobutyric acid (GABA), L-Alanine, L-Glutamic acid, all as free amino acids. The composition can include a therapeutically effective derivative of these amino acids, such as, but not limited to, a salt or an N-acetyl form. The composition can include a therapeutically effective metabolite of these amino acids. In one embodiment, the metabolite is 5-Hydroxytryptophan. Thus, in several non-limiting examples, 5-Hydroxytryptophan is included in the composition, such that the composition includes only 5-Hydroxytrytophan (without L-Tryptophan) or a mixture of L-Tryptophan and 5-Hydroxytryptophan.

Generally, the composition also includes least one B vitamin. The B vitamin can be vitamin B2, vitamin B3, vitamin B6 or vitamin B12. More than one B vitamin can also be included in the composition. Thus, the composition can include two, three or all four of vitamin B2, vitamin B3, vitamin B6 and vitamin B12. In one example, the composition includes about 0.1 to about 0.5 percent by weight coenzyme vitamin B2. In another example, the composition includes about 0.1 to about 0.5 percent by weight Vitamin B3. In a further example, the composition includes about 0.1 to about 0.5 percent by weight Vitamin B6. In yet another example, the composition includes about 0.0001 about 0.0003 percent by weight Vitamin B12. The composition can include more than one of vitamin B2, vitamin B3, vitamin B6 and vitamin B12 at these concentrations. Thus, the composition can include about 0.1 to about 0.5 percent by weight coenzyme vitamin B2, about 0.1 to about 0.5 percent by weight Vitamin B3, about 0.1 to about 0.5 percent by weight Vitamin B6, and/or about 0.0001 about 0.0003 percent by weight Vitamin B12. In one example, the composition includes vitamin B2, vitamin B3, vitamin B6 and vitamin B12.

In several examples, the composition includes one or more of: 5-6 percent by weight of L-Tyrosine, 4-6 percent by weight L-Carnitine, and 3-4 percent by weight GABA or 6-7 percent by weight L-Tryptophan. Thus, the composition can include 5-6 percent by weight of L-tyrosine, 4-6 percent by weight L-Carnitine, and 3-4 percent by weight GABA and 6-7 percent by weight L-Tryptophan (or 5-Hydroxytryptophan or a mixture thereof).

The composition can include additional active ingredients, such as folic acid, and magnesium. In one example, the composition includes about 0.001 to about 0.003 percent by weight folic acid. In another example, the composition includes about 2 to about 3 percent by weight magnesium. Thus, the composition can include about 0.001 to about 0.003 percent by weight folic acid and about 2 to about 3 percent by weight magnesium as an amino acid chelate.

In one example, the composition includes the following ingredients shown in Table 1, either as Composition I or Composition II. In Table I, the amounts are expressed as percent by weight in a pharmaceutically acceptable carrier.

TABLE 1

| Agent | Composition I | Composition II |
|---|---|---|
| L-Lysine | 5-7% | 6-7% |
| L-Valine | 5-7% | 6-7% |
| L-Tryptophan* | 5-7% | 6-7% |
| L-Phenylalanine | 4-6% | 5-6% |
| L-Methionine | 4-6% | 4-5% |
| L-Leucine | 3-5% | 4-5% |
| L-Threonine | 3-5% | 4-5% |
| L-Isoleucine | 3-5% | 3-4% |
| L-Arginine | 2-4% | 3-4% |
| L-Histidine | 2-4% | 3-4% |
| L-Tyrosine | 4.5%-6.5% | 5-6% |
| L-Carnitine | 4-6% | 4.5-6% |
| L-Serine | 4-6% | 4.5-6% |
| L-Glutamine | 4-6% | 4.5-6% |
| Aspartic Acid | 3-5% | 4-5% |
| L-Proline | 2.5-4.5% | 3-4% |
| L-Glycine | 2.5-4.5% | 3-4% |
| Taurine | 2.5-4.5% | 3-4% |
| L-Cysteine | 2.5-4.5% | 3-4% |
| (as N-Acetyl Cysteine) | | |
| GABA | 2-4% | 3-4% |
| L-Alanine | 2-4% | 3-4% |
| L-Glutamic acid | 2-4% | 3-4% |

(*or 5-Hydroxytryptophan or a mixture thereof)

The composition can also include an effective amount of Vitamin B2, Vitamin B3, Vitamin B6, Vitamin B12, Folic Acid, and/or magnesium, or any combination thereof. In one embodiment, the composition includes the free amino acids listed in Table 1, and Vitamin B2, Vitamin B3, Vitamin B6, Vitamin B12, Folic Acid, and magnesium (such as an amino acid chelate). Amino acid chelates of magnesium are well known in the art, and are commercially available. For example, a magnesium amino acid chelate can be Magnesium Oxide bonded to a mixture of ligands, including Glycine, Aspartic Acid and Citric Acid. Magnesium amino acid chelates are available commercially such as through Monarch Nutritional Laboratories, Science Lab and Albion Advanced Nutrition. However, an equivalent amount of elemental magnesium or a magnesium salt could also be included.

In one example, the composition includes about 0.001 to about 0.003 percent by weight folic acid. The composition can also include about 1 to about 3 percent magnesium as an amino acid chelate, such as about 1.5 to 2.5 percent magnesium as an amino acid chelate by weight, such as about 2 percent magnesium as an amino acid chelate by weight. The composition can include an equivalent amount elemental magnesium in any form. Thus, in additional example, the composition includes magnesium salts. Thus, the composition can include about 0.001 to about 0.003 percent by weight folic acid and about 2 to about 3 percent by weight magnesium as an amino acid chelate. In several examples, the composition includes 10 to 20 mg of magnesium as an amino acid chelate. In additional example, the amino acid chelate of magnesium is not included in the calculation of the percent by weight of a free amino acid.

It should be noted that additional ingredients can be included, such as vitamins and minerals. Thus, vitamin C can be included in the composition. Additional vitamins and minerals, such as vitamin A or zinc, can further be included in the composition. Proteins such as enzymes can also be included in the composition.

In a further embodiment, the composition can include the following ingredients listed in Table 2 as percent by weight. The amounts can be approximately the amounts listed in Table 2, or can be exact percentage listed in Table 2.

TABLE 2
Essential Amino Acids

| | |
|---|---|
| L-Lysine | 6.25% |
| L-Valine | 6.25% |
| L-Tryptophan* | 6.25% |
| L-Phenylalanine | 5.625% |
| L-Methionine | 5.0% |
| L-Leucine | 4.375% |
| L-Threonine | 4.375% |
| L-Isoleucine | 3.75% |
| L-Arginine | 3.125% |
| L-Histidine | 3.125% |

Non-Essential Amino Acids

| | |
|---|---|
| L-Tyrosine | 5.5% |
| L-Carnitine | 5.0% |
| L-Serine | 5.0% |
| L-Glutamine | 5.0% |
| Aspartic Acid | 4.375% |
| L-Proline | 3.75% |
| L-Glycine | 3.75% |
| Taurine | 3.75% |
| L-Cysteine (as N-Acetyl Cysteine) | 3.75% |
| GABA | 3.125% |
| L-Alanine | 3.125% |
| L-Glutamic acid | 3.125% |
| Coenzyme Vitamin B2 (Flavin mononucleotide/Riboflavin) | 0.125% |
| Vitamin B3 (Niacinamide) | 0.125% |
| Vitamin B6 (Pyridoxal 5-Phosphate) | 0.125% |
| Vitamin B12 (Methylacobalamin) | 0.00025% |
| Folic Acid (Calcium Folinate) | 0.00166% |
| Magnesium (Amino acid chelate) | 2.0% |

*or 5-Hydroxytrytophan or a mixture thereof

The composition can include the following ingredients listed in Table 3 as percentage by weight:

TABLE 3
Essential Amino Acids

| | |
|---|---|
| L-Lysine | 6.21% |
| L-Valine | 6.21% |
| L-Tryptophan* | 6.21% |
| L-Phenylalanine | 5.59% |
| L-Methionine | 5.0% |
| L-Leucine | 4.35% |
| L-Threonine | 4.35% |
| L-Isoleucine | 3.73% |
| L-Arginine | 3.11% |
| L-Histidine | 3.11% |

Non-Essential Amino Acids

| | |
|---|---|
| L-Tyrosine | 5.5% |
| L-Carnitine | 5% |
| L-Serine | 5% |
| L-Glutamine | 5.% |
| Aspartic Acid | 4.35% |
| L-Proline | 3.73% |
| L-Glycine | 3.73% |
| Taurine | 3.73% |
| L-Cysteine (as N-Acetyl Cysteine) | 3.73% |
| GABA | 3.11% |
| L-Alanine | 3.11% |
| L-Glutamic acid | 3.11% |
| Coenzyme Vitamin B2 (Flavin | 0.14% |
| mononucleotide/Riboflavin) | |
| Vitamin B3 (Niacinamide) | 0.14% |
| Vitamin B6 (Pyridoxal 5-Phosphate) | 0.14% |
| Vitamin B12 (Methylacobalamin) | 0.00025% |
| Folic Acid (Calcium Folinate) | 0.00186% |
| Magnesium (Amino acid chelate) | 1.98% |

*or 5-Hydroxytryptophan or a mixture thereof

In yet a further embodiment, one or more flavoring ingredients can be added to the composition to improve its palatability, taste, and/or its odor. Flavoring compounds, which can be added, include orange flavoring, vanilla, citrus, cherry, lemon, lime, chocolate, coffee, strawberry, mint, banana, bubble gum, cinnamon, pumpkin, apple, blueberry, pineapple, blackberry, kiwi, and raspberry. In several examples, the flavor is orange, vanilla, or citrus. However, many other such flavoring ingredients could also be chosen. Suitable flavorant additives which exhibit flavor and aroma enhancing properties generally are organic compounds which correspond to structure classifications such as aliphatic and aromatic alcohols, furan ethers, thiazole alcohols, pyridine ethers and alcohols, benzofuran carbonyl compounds, aliphatic and aromatic ketones, alpha-diketones, pyrrole-alpha-diketones, aromatic sulfur compounds, phenols and phenol ethers, and the like, for example see U.S. Pat. No. 3,702,253. Flavorant additives are illustrated by compounds such as anethole, benzaldehyde, bergamot oil, acetoin, carvol, cinnamaldehyde, citral, ethylvanillin, vanillin, thymol, methyl salicylate, coumarin, anise, cinnamon, ginger, clove, lemon oil, 1-undecanol, 5-dodecalactone, eugenol, geraniol, geranyl acetate, guaiacol, limonene, linalool, piperonal, 2-acetyl-5-methylpyrazine, 2-ethyl-3-methoxypyrazine, 5-methylquinoxaline, 2-methyl-6-propylpyrazine, 2-methylbenzofuran, 2,2′-dithienylmethane, benzyl hexyl carbinol, furfuryl phenyl ether, difurfuryl ether, benzofuran-2-aldehyde, benzothiophene-2-aldehyde, 1-butylpyrrole-2-aldehyde, methyl decyl ketone, dipropyl ketone, ethyl benzyl ketone, 2,6-diacetylpyridine, heptane-3,4-dione, methyl thiophene-2-carboxylate, 2-hydroxyacetophenone, 4-ethyl-2-methoxyphenol, 2-oxobutan-1-ol, and the like. Any flavoring agent or ingredient that is known in the art may be employed in the compositions disclosed herein.

The amino acid compositions disclosed herein can be administered in any form, including as solids such as tablets or powders or as a liquid preparation. In one example, the compositions are formulated for enteral administration. An example of a formulation of use is a pharmaceutical preparation (such as a tablet, enteral liquid, parenteral liquid, capsule, intranasal liquid or other form). In a particular disclosed example the supplement is a pharmaceutical preparation, in particular a tablet or capsule.

Thus, the composition can contain a suitable pharmaceutical carrier or diluent, and optionally contain a stabilizer, a pH adjusting agent, and other additives. The pH of the preparation can be adjusted to 3.0-8.0, preferably 4.0-7.0, if the preparation is administered in a liquid form.

Compositions suitable for oral administration may be presented as discrete units such as capsules, cachets, or tablets, each containing a therapeutically effective amount of the composition, as a powder or granules, or as a solution or a suspension in an aqueous liquid, a non-aqueous liquid, an oil-in-water emulsion, or a water-in-oil liquid emulsion. In general, the compositions are prepared by homogenously admixing the active ingredients with liquid carriers or finely divided solid carriers or both, and then, if necessary, shaping the product into the desired presentation.

The compositions can additionally include inactive ingredients such as binding agents (such as pregelatinized maize starch, polyvinylpyrrolidone or hydroxypropyl methylcellulose); binders or fillers (such as lactose, pentosan, microcrystalline cellulose or calcium hydrogen phosphate); lubricants (such as magnesium stearate, talc or silica); disintegrants (such as potato starch or sodium starch glycolate); or wetting agents (such as sodium lauryl sulphate). The composition can include magnesium sterate, such as about 0.5 to about 2 percent by weight magnesium sterate, such about 0.5 to 1 percent by weight magnesium sterate, such as 0.75 percent by weight magnesium sterate.

In one example, a tablet containing the compositions disclosed herein can be prepared by compression or molding, optionally, with one more accessory ingredients. Compressed tablets can be prepared by compressing in a suitable machine free amino acids a free-flowing form such as powder or granules, optionally mixed with a binder, lubricant, inert diluent, surface active or dispersing agent. The composition, such as the tablet, can include pharmaceutically acceptable components such as lactose, glucose, sucrose, corn starch, potato starch, cellulose esters such as cellulose acetate, ethyl cellulose, magnesium stearate, calcium silicate, precipitated silica, talc, fatty acids such as stearic acid, microcrystalline cellulose, carnauba wax and the like. The tablets or capsules can be coated by methods well known in the art.

Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives that are inactive agents, such as suspending agents (such as sorbitol syrup, cellulose derivatives or hydrogenated edible fats), emulsifying agents (such as lecithin or acacia), nonaqueous vehicles (such as almond oil, oily esters, ethyl alcohol or fractionated vegetable oils), and preservatives (such as methyl or propyl-p-hydroxybenzoates or sorbic acid). The compositions can also be made to be pleasant tasting, and thus can contain buffer salts, flavoring, coloring and sweetening agents as appropriate.

Thus, dosage forms include tablets, capsules, dispersions, suspensions, solutions, capsules and the like. Because of their ease of administration, tablets and capsules represent a convenient oral dosage unit form, in which case solid pharmaceutical carriers as described above are employed. However, in addition to the common dosage forms set out above, the compounds can also be administered by controlled release means, or can be formulated for other means of delivery, such as, but not limited to intranasal or transdermal delivery.

Diluents and other inactive ingredients such as one or more pharmaceutically acceptable binding agents, fillers, supports, thickening agents, taste-improving agents, coloring agents, preservatives, stabilizers, regulators, emulsifiers, flow agents, absorbents, and the like or mixtures thereof may be used depending on the form of the composition employed. The composition can also include a sweetener, such as a natural (for example, sugar or honey) or artificial sweetener (for example, saccharine), if desired. Generally, the carriers, sugars, diluents, stabilizers, buffers, flavoring and texturing ingredients are considered to be inactive ingredients, as they do not impart a therapeutic effect in and of themselves.

The administered compositions can be made to have any desired amount of calories. In some examples, the compositions contain from about 1 to about 100 calories, or from about 100 to about 500 calories, such as about 100, about 150, about 200 or about 300 calories. In particular instances the compositions contain no more than about 200 calories, or no more than 350 calories.

Generally, the compositions described herein can be administered to any subject, including healthy subjects. The composition can be used in methods for treating a human afflicted by a variety of diseases, conditions, and symptoms that associated with a deficiency of an amino acid. In addition to treatment of the disease, the methods of the invention can also be used for preventive treatment in a person susceptible to such diseases, disorders or symptoms.

In several embodiments, a therapeutically effective amount of the composition is administered to a subject that has a condition associated with a relative deficiency of one or more amino acids, including conditions such as, but not limited to, soft tissue injury, insomnia, panic attacks, anxiety disorders, eating disorders, anorexia, depression, chronic pain, emotional distress, chemical dependence, alcoholism, and hypoglycemia. The compositions can be administered in a variety of dosing regimens to achieve the desired therapeutic effect, such as to achieve a reduction in a sign or a symptom of a conditional associated with a deficiency of one or more amino acids. One of skill in the art will readily be able to determine a dosing protocol for optimal effects by monitoring the signs and/or symptoms of the disorder.

In several embodiments, the composition can be administered one, two or three times a day. The composition can also be administered every two, three, four, five six or seven days. Generally, repeated administration is contemplated, but in some instances a single administration may result in the desired therapeutic effect. Intermittent administration can also be used, wherein the composition is administered to the subject only sporadically with a specified time period between dosages. Thus, in some examples, there would be a one-day interval, such as a two or a three-day interval between any two days on which the composition is administered to the subject. Administration can be continued so long as the desired effect is achieved. Thus, administration over a period of days, weeks, months or even years is contemplated.

### EXAMPLES

The following examples are provided to illustrate particular features of various described embodiments. The scope of the present invention should not be limited to those features exemplified.

### Example 1

### Exemplary Formulations

Capsules containing the following formulation were produced (mg=milligrams, mcg—micrograms):

| | | | Specific | |
|---|---|---|---|---|
| Agent | Range | | Amount | |
| L-Lysine (as L-lysine hydrochloride) | 45-55 | mg | 50 | mg |
| L-Valine | 45-55 | mg | 50 | mg |
| L-Tryptophan | 45-55 | mg | 50 | mg |
| L-Phenylalanine | 35-50 | mg | 45 | mg |
| L-Methionine | 35-45 | mg | 40 | mg |
| L-Leucine | 30-40 | mg | 35 | mg |
| L-Threonine | 30-40 | mg | 35 | mg |
| L-Isoleucine | 25-35 | mg | 30 | mg |
| L-Arginine | 20-30 | mg | 25 | mg |
| L-Histidine (as L-histidine hydrochloride) | 20-30 | mg | 25 | mg |
| L-Tyrosine | 40-50 | mg | 44 | mg |
| L-Carnitine | 35-45 | mg | 40 | mg |
| L-Serine | 35-40 | mg | 40 | mg |
| L-Glutamine | 35-40 | mg | 40 | mg |
| Aspartic Acid | 30-40 | mg | 35 | mg |
| L-Proline | 25-35 | mg | 30 | mg |
| L-Glycine | 25-35 | mg | 30 | mg |
| Taurine | 25-35 | mg | 30 | mg |
| L-Cysteine (as N-Acetyl Cysteine) | 25-35 | mg | 30 | mg |
| Gamma aminobutyric acid (GABA) | 20-30 | mg | 25 | mg |
| L-Alanine | 20-30 | mg | 25 | mg |
| L-Glutamic acid | 20-30 | mg | 25 | mg |
| Coenzyme Vitamin B2 | 0.5-1.5 | mg | 1 | mg |
| (Flavin mononucleotide/Riboflavin) | | | | |
| Vitamin B3 (Niacinamide) | 0.5-1.5 | mg | 1 | mg |
| Vitamin B6 (Pyridoxal 5-Phosphate) | 0.5-1.5 | mg | 1 | mg |
| Vitamin B12 (Methylacobalamin) | 10-30 | mcg | 20 | mcg |
| Folate (Calcium Folinate) | 100-150 | mcg | 133.3 | mcg |
| Magnesium (Amino acid chelate) | 10-20 | mg | 16 | mg |

Magnesium Stearate (6.13 mg) was also included; the total weight of ingredients in each capsule was 805 mg. Clear capsules, size “00” were used filled with the composition. Packages were produced that included 180 capsules per 400 cc in a bottle with a white induction lined cap, neck shrink band.

Capsules containing the following formulation were also produced:

| | Specific | |
|---|---|---|
| Agent | Amount | |
| L-Lysine (as L-lysine hydrochloride) | 50 | mg |
| L-Valine | 50 | mg |
| L-Tryptophan | 50 | mg |
| L-Phenylalanine | 45 | mg |
| L-Methionine | 40 | mg |
| L-Leucine | 35 | mg |
| L-Threonine | 35 | mg |
| L-Isoleucine | 30 | mg |
| L-Arginine | 25 | mg |
| L-Histidine (as L-histidine hydrochloride) | 25 | mg |
| L-Tyrosine | 44 | mg |
| L-Carnitine L-Tartrate | 40 | mg |
| L-Serine | 40 | mg |
| L-Glutamine | 40 | mg |
| Aspartic Acid | 35 | mg |
| L-Proline | 30 | mg |
| L-Glycine | 30 | mg |
| Taurine | 30 | mg |
| L-Cysteine (as N-Acetyl Cysteine) | 30 | mg |
| Gamma aminobutyric acid (GABA) | 25 | mg |
| L-Alanine | 25 | mg |
| L-Glutamic acid | 25 | mg |
| Coenzyme Vitamin B2 | 1.1 | mg |
| (Flavin mononucleotide/Riboflavin) | | |
| Vitamin B3 (Niacinamide) | 1.1 | mg |
| Vitamin B6 (Pyridoxal 5-Phosphate) | 1.5 | mg |
| Vitamin B12 (Methylacobalamin) | 21 | mcg |
| Folate (Calcium Folinate) | 150 | mcg |
| Magnesium (Amino acid chelate) | 16 | mg |

Magnesium Stearate (6.13 mg) was also included; the total weight of ingredients in each capsule was 805 mg. In this example, the weight of the amino acids that are included in the amino acid chelate of magnesium is not included in the listed weights of free amino acids (magnesium amino acid chelate is excluded when the amount/percent of amino acids is calculated).

### Example 2

### Case Report for Tissue Repair

A subject with a history of fibromyalgia is administered the composition described in example 1 daily for a period of several months. Observations of clinical symptoms of allergies, intense muscle pain associated with fibromyalgia, and microbial forms associated with red blood cells were conducted. A correlation exists between parasitized red blood cells and fibromyalgia/chronic fatigue syndrome. See, e.g., Nasraiia, M., et al., Eur. J. Clin. Microbiol. Infect. Dis., 18(12):859-65 (1999); Tarello, W., Comp. Immunol. Microbiol. Infect. Dis. 24(1):57-70 (2001); Vojdani A., et al., FEMS Immunol. Med. Microbiol., 22(4):355-65 (1998); and Choppa, P. C., et al., Mol. Cell. Probes, 12(5):301-08 (1998).

Over the observation period, consumption of the composition correlated to a decrease in symptoms and an increased proportion of non-parasitized red blood cells versus parasitized red blood cells, thus improving delivery of oxygen to muscle tissue and reducing muscle pain.

### Example 3

### Treatment for Depression

A subject with a clinical history of a moderately severe chronic depression is treated with the composition described in Example 1. During the past years the subject has undergone treatment with various combinations of chemical agents, such as tricyclic antidepressants, as prescribed by her psychiatrist. The subject is evaluated by the Beck Depression Inventory (1996, Harcourt). The pretreatment score is indicative of an ongoing major clinical depression.

The subject is treated with the composition described in Example 1 twice daily for a period of a month. At follow-up the subject reports a perceived benefit in terms of mood. Objective evaluation reveals a follow-up score of 5 on the Beck's scale, indicating that the subject is no longer clinically depressed.

### Example 4

### Treatment for Anxiety and Panic Attacks

A subject with a history of intermittent anxiety and panic for ten years, whose medications includes ZOLOFT™, is treated with the compositions described herein, such as the composition of Example 1. Prior to treatment she reports panic and anxiety attacks. The composition is administered twice a day over a period of months. Months later the subject returns for a follow-up appointment, and reports an absence of panic and anxiety attacks.

### Example 5

### Treatment for Chemical Dependence

A subject with a history of alcoholism is treated with the compositions described herein, such as the composition of Example 1. The composition is administered daily over a period of one to six months. The subject receives simultaneous psychological counseling. Months later the subject returns for a follow-up appointment, and reports that they have not imbibed alcohol during the treatment period.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A nutritional composition comprising a homogenous mixture of free amino acids, wherein the homogenous mixture of free-form amino acids comprises L-Lysine, L-Valine, L-Tryptophan or a metabolite thereof, L-Phenylalanine, L-methionine, L-Leucine, L-Threonine, L-Isoleucine, L-Arginine, L-Histindine, L-Tyrosine, L-Carnitine, L-Serine, L-Glutamine, Aspartic Acid, L-Proline, L-Glycine, Taurine, L-Cysteine, Gamma-aminobutyric acid (GABA), L-Alanine, and L-Glutamic acid,
and wherein the composition further comprises at least one B vitamin.

2. The nutritional composition of claim 1, wherein the B vitamin is vitamin B2, vitamin B3, vitamin B6, folic acid or vitamin B12.

3. The nutritional composition of claim 1, comprising vitamin B2, vitamin B3, vitamin B6, folic acid and vitamin B12.

4. The nutritional composition of claim 3, further comprising magnesium.

5. The nutritional composition of claim 1, comprising one or more of:
(a) 4.5-6.5 percent by weight of L-Tyrosine;
(b) 4-6 percent by weight L-Carnitine;
(c) 2-4 percent by weight GABA;
(d) 5-7 percent by weight L-Tryptophan;
(e) about 0.1 to about 0.5 percent by weight coenzyme vitamin B2;
(f) about 0.1 to about 0.5 percent by weight Vitamin B3;
(g) about 0.1 to about 0.5 percent by weight Vitamin B6;
(h) about 0.0001 to about 0.0003 percent by weight Vitamin B12;
(i) about 0.001 to about 0.003 percent by weight folic acid; and
(j) about 2 to about 3 percent by weight of magnesium as an amino acid chelate.

6. The nutritional composition of claim 1, comprising about 2 to about 3 percent by weight of magnesium as an amino acid chelate.

7. The nutritional composition of claim 1, comprising the following ingredients as percent by weight:

| | |
|---|---|
| L-Lysine | 5-7% |
| L-Valine | 5-7% |
| L-Tryptophan | 5-7% |
| L-Phenylalanine | 4-6% |
| L-Methionine | 4-6% |
| L-Leucine | 3-5% |
| L-Threonine | 3-5% |
| L-Isoleucine | 3-5% |
| L-Arginine | 2-4% |
| L-Histidine | 2-4% |
| L-Tyrosine | 4.5%-6.5% |
| L-Carnitine | 4-6% |
| L-Serine | 4-6% |
| L-Glutamine | 4-6% |
| Aspartic Acid | 3-5% |
| L-Proline | 2.5-4.5% |
| L-Glycine | 2.5-4.5% |
| Taurine | 2.5-4.5% |
| N-Acetyl L-Cysteine | 2.5-4.5% |
| GABA | 2-4% |
| L-Alanine | 2-4% |
| L-Glutamic acid | 2-4% |

and a pharmaceutically acceptable carrier.

8. The nutritional composition of claim 7, comprising the following ingredients as percent by weight:

| | |
|---|---|
| L-Lysine | 6-7% |
| L-Valine | 6-7% |
| L-Tryptophan | 6-7% |
| L-Phenylalanine | 5-6% |
| L-Methionine | 4-5% |
| L-Leucine | 4-5% |
| L-Threonine | 4-5% |
| L-Isoleucine | 3-4% |
| L-Arginine | 3-4% |
| L-Histidine | 3-4% |
| L-Tyrosine | 5-6% |
| L-Carnitine | 4.5-6% |
| L-Serine | 4.5-6% |
| L-Glutamine | 4.5-6% |
| Aspartic Acid | 4-5% |
| L-Proline | 3-4% |
| L-Glycine | 3-4% |
| Taurine | 3-4% |
| L-Cysteine | 3-4% |
| GABA | 3-4% |
| L-Alanine | 3-4% |
| L-Glutamic acid | 3-4% |

9. The nutritional composition of claim 1, further comprising an effective amount of Coenzyme Vitamin B2, Vitamin B3, Vitamin B6, Vitamin B12, Folic Acid, and Magnesium.

10. The nutritional composition of claim 1, comprising a percent by weight of about:

| | |
|---|---|
| L-Lysine | 6.25% |
| L-Valine | 6.25% |
| L-Tryptophan | 6.25% |
| L-Phenylalanine | 5.625% |
| L-Methionine | 5.0% |
| L-Leucine | 4.375% |
| L-Threonine | 4.375% |
| L-Isoleucine | 3.75% |
| L-Arginine | 3.125% |
| L-Histidine | 3.125% |
| L-Tyrosine | 5.5% |
| L-Carnitine | 5.0% |
| L-Serine | 5.0% |
| L-Glutamine | 5.0% |
| Aspartic Acid | 4.375% |
| L-Proline | 3.75% |
| L-Glycine | 3.75% |
| Taurine | 3.75% |
| N-Acetyl L-Cysteine | 3.75% |
| GABA | 3.125% |
| L-Alanine | 3.125% |
| L-Glutamic Acid | 3.125% |
| Coenzyme Vitamin B2 | 0.125% |
| Vitamin B3 | 0.125% |
| Vitamin B6 | 0.125% |
| Vitamin B12 | 0.00025% |
| Folic Acid | 0.00166% |

11. The composition of claim 1, comprising:

| | |
|---|---|
| L-Lysine | 6.21% |
| L-Valine | 6.21% |
| L-Tryptophan | 6.21% |
| L-Phenylalanine | 5.59% |
| L-Methionine | 5.0% |
| L-Leucine | 4.35% |
| L-Threonine | 4.35% |
| L-Isoleucine | 3.73% |
| L-Arginine | 3.11% |
| L-Histidine | 3.11% |
| L-Tyrosine | 5.5% |
| L-Carnitine | 5% |
| L-Serine | 5% |
| L-Glutamine | 5.% |
| Aspartic Acid | 4.35% |
| L-Proline | 3.73% |
| L-Glycine | 3.73% |
| Taurine | 3.73% |
| N-Acetyl L-Cysteine | 3.73% |
| GABA | 3.11% |
| L-Alanine | 3.11% |
| L-Glutamic acid | 3.11% |
| Coenzyme Vitamin B2 | 0.14% |
| Vitamin B3 | 0.14% |
| Vitamin B6 | 0.14% |
| Vitamin B12 | 0.00025% |
| Folic Acid | 0.00186%. |

12. The composition of claim 10, further comprising about 2 percent by weight magnesium.

13. The nutritional composition of claim 1, formulated for enteral administration.

14. The nutritional composition of claim 13, formulated for oral administration.

15. The nutritional composition of claim 13, wherein formulated as a solid oral dosage form.

16. The nutritional composition of claim 13, further comprising a pharmaceutically effective carrier, a flavoring agent, a sweetening agent, or a combination thereof.

17. A composition, comprising:

| | |
|---|---|
| L-lysine hydrochloride | 45-55 mg |
| L-Valine | 45-55 mg |
| L-Tryptophan | 45-55 mg |
| L-Phenylalanine | 35-45 mg |
| L-Methionine | 35-45 mg |
| L-Leucine | 30-40 mg |
| L-Threonine | 30-40 mg |
| L-Isoleucine | 25-35 mg |
| L-Arginine | 20-30 mg |
| L-histidine hydrochloride | 20-30 mg |
| L-Tyrosine | 40-50 mg |
| L-Carnitine | 35-45 mg |
| L-Serine | 35-40 mg |
| L-Glutamine | 35-40 mg |
| Aspartic Acid | 30-40 mg |
| L-Proline | 25-35 mg |
| L-Glycine | 25-35 mg |
| Taurine | 25-35 mg |
| N-Acetyl Cysteine | 25-35 mg |
| Gamma aminobutyric acid (GABA) | 20-30 mg |
| L-Alanine | 20-30 mg |
| L-Glutamic acid | 20-30 mg |

and a pharmaceutically acceptable carrier.

18. The composition of claim 17, further comprising:

| | | |
|---|---|---|
| Coenzyme Vitamin B2 | 0.5-1.5 | mg |
| Vitamin B3 | 0.5-1.5 | mg |
| Vitamin B6 | 0.5-1.5 | mg |
| Vitamin B12 | 10-30 | mcg |
| Folate | 100-150 | mcg |
| Magnesium, amino acid chelate | 10-20 | mg |

19. The composition of claim 17, comprising:

| | |
|---|---|
| L-lysine hydrochloride | 50 mg |
| L-Valine | 50 mg |
| L-Tryptophan | 50 mg |
| L-Phenylalanine | 45 mg |
| L-Methionine | 40 mg |
| L-Leucine | 35 mg |
| L-Threonine | 35 mg |
| L-Isoleucine | 30 mg |
| L-Arginine | 25 mg |
| L-histidine hydrochloride | 25 mg |
| L-Tyrosine | 44 mg |
| L-Carnitine | 40 mg |
| L-Serine | 40 mg |
| L-Glutamine | 40 mg |
| Aspartic Acid | 35 mg |
| L-Proline | 30 mg |
| L-Glycine | 30 mg |
| Taurine | 30 mg |
| N-Acetyl Cysteine | 30 mg |
| Gamma aminobutyric acid (GABA) | 25 mg |
| L-Alanine | 25 mg |
| L-Glutamic acid | 25 mg |

and a pharmaceutically acceptable carrier.

20. The composition of claim 19, further comprising:

| | | |
|---|---|---|
| Vitamin B2 | 1.1 | mg |
| Vitamin B3 | 1.1 | mg |
| Vitamin B6 | 1.5 | mg |
| Vitamin B12 | 20 | mcg |
| Folate | 150 | mcg |
| Magnesium | 16 | mg |

and optionally a lubricant.

21. The composition of claim 19, wherein the lubricant is magnesium stearate.

22. A method of ameliorating a condition associated with a relative deficiency of one or more amino acids in a subject, comprising administering to the subject a physiologically effective amount of the composition of claim 1, thereby ameliorating the condition.

23. The method of claim 22, wherein the condition a soft tissue injury, an eating disorder, anorexia, a mood disorder, depression, anxiety, chronic pain, emotional distress, chemical dependence, alcoholism, hypoglycemia, a panic disorder, insomnia, fibromyalgia or a combination thereof.
